(19) United States
(12) Patent Application Publication
(10) Pub. No.: US 2022/0017924 A1
(43) Pub. Date: Jan. 20, 2022

(54) Cell Microporator

(71) Applicant: UAB "FEMTIKA", Vilnius (LT)

(72) Inventors: Linas JONUSAUSKAS, Vilnius (LT); Roaldas GADONAS, Vilnius (LT)

(73) Assignee: UAB "FEMTIKA", Vilnius (LT)

(21) Appl. No.: 17/375,851

(22) Filed: Jul. 14, 2021

(30) Foreign Application Priority Data
Jul. 14, 2020 (LT) 2020 027

(51) Int. Cl. C12N 15/87

(52) U.S. Cl. CPC C12N 15/87

(57) ABSTRACT

A cell microporator containing a fluid microchannel, having elements of mechanical action on cells is provided. The cell microporator comprises mechanically acting elements that are made as two rotating rollers, one of which has a smooth composing surface and the other has teeth. A gap between the constituent surfaces of the two rollers is smaller than the size of the cells to be treated.

### CROSS-REFERENCES TO RELATED APPLICATIONS

This application claims priority from Lithuanian Application Serial Number 2020 027 filed Jul. 14, 2021, which is hereby incorporated herein by reference in its entirety.

### TECHNICAL FIELD

The invention falls within the field of biomedicine, more specifically to cell transfection devices.

### BACKGROUND OF THE INVENTION

Transfection is the technique of introducing alien genes into cells. The cell membrane protects the interior of the cell from the entry of foreign biological material, so transfection must temporarily disrupt this barrier function of the membrane.
Various physical methods of transfection are known and used:
electroporation where the cell is exposed to an electric field for a short time;
cell squeezing and close hydrodynamic transfection of cells;
sonoporation—the effect of cells by ultrasound;
optical transfection, where a hole of about 1 micron is made in a cell membrane by means of with a particularly well-focused laser beam;
various micro-injection techniques, such as the introduction of foreign material using micro glass tubes or the use of a gene cannon.

The essence of all the above methods is that temporary pores or holes are created in the cell membrane through which desired extraneous substances, inorganic compounds, medicines, genetic materials, proteins, carbohydrates, synthetic polymer and pharmaceutical compositions are introduced into the cell.

Patent US2020063163 describes the methods and sets of nozzles for introducing a substance into cells. Cells that move through a microchannel filled with liquid or via a passageway increase the speed at the microchannel narrowing by passing through a narrower diameter outlet. The pressure in cells suddenly changes from high to low. Pressure changes (from high to low) results in temporary stretching of the cells, forming temporary holes in cell membranes that allow the entry of substances contained in the fluid or solution surrounding the cell, including the genetic material cDNA, SiRNA, and miRNA. The low concentration of Ca 2+ in the liquid or solution stimulates extending the time needed for the cell membranes to be sealed, thereby encouraging the entry of the substance into the cell.

The US2019262835 invention presents a method of transfection, characterised by multiple compression and release of cells flowing via a fluid microchannel with a stepped cross-sectional area. As cells are passed through the compression interval, the inner volume of cells (Vloss) decreases (Vloss) and when they enter the release area, the volume thereof increases (“Vgain”)—they absorb the part of the molecules present in the liquid.

There is a known method of cell transfection (see patent EP3556845) in which pairs of cells result from the formation of obstructions in a microchannel, e.g. due to a sudden change in the cell flow vector.

In all the above-listed inventions, cell deformation is performed by a sudden change in the pressure on the cells due to a change in the shape of the microchannel. The disadvantage of these inventions is that cells moving in a microchannel are in most cases exposed to slip friction—they are towed along the walls of the microchannel.

There is a known microporator of cells (see patent U.S. Pat. No. 6,846,668). It has a tube with an inlet and an outlet for transferring the cells suspended in fluid and an injection area with an injection needle projecting from the wall, designed to puncture cells through which the desired substance is injected into the cell or the substance is suck out from the cell. This can be done in any order desired number of times.

This invention uses a mechanical method of their transfection they are punctured with a microneedle. In order to increase cell transfusion efficiency, multiple microchannels need to be mounted in parallel.

### BRIEF SUMMARY OF EMBODIMENTS OF THE INVENTION

The objective of this invention is to offer the part of an efficient cell transfection device—a microporator, which can be attributed to mechanical means of transfection. The proposed microporator includes three methods of cell transfection:
mechanical deformation of cells (compression and release),
puncture of the cell membrane,
mixed mode (deformation and puncture).

In order to achieve the purpose of the invention, the microporator consisting of a fluid microchannel and the elements of mechanical action placed therein, the latter was made as two rotating rollers (synonym ‘rolls’) and mounted in recesses of the microchannel at a distance from each other less than the size of the cells to be processed, one or both rollers made with teeth or spikes, the roller being passive, rotated by flowing fluid and are in contact with the cells on one side.

Various roller-shaped options are offered: cylindrical, convex-concave (pair), concave, stepped cylindrical. The following options are offered for mounting the camshafts: the rollers have recesses at the end and protrusions (short axles) in the fluid microchannel and vice versa—the rollers have recesses in the ends and protrusions (short axles) in the fluid microchannel, which serve as slide bearings, or the rollers are mounted on axles.

The microporator may have several pairs of rollers.

### DESCRIPTION OF THE DRAWINGS

The invention is illustrated by drawings.

The drawings show the cross-sections of the microporator in which the elements of mechanical impact are pairs of rollers:

FIG. 1—one roller with a smooth cylindrical surface, the other one with teeth,

FIG. 2—both rollers with teeth,

FIG. 3—one roller with a smooth cylindrical surface, the other one with spikes,

FIG. 4—both rollers with spikes.

### DESCRIPTION OF THE DESIGN OF THE MICROPORATOR

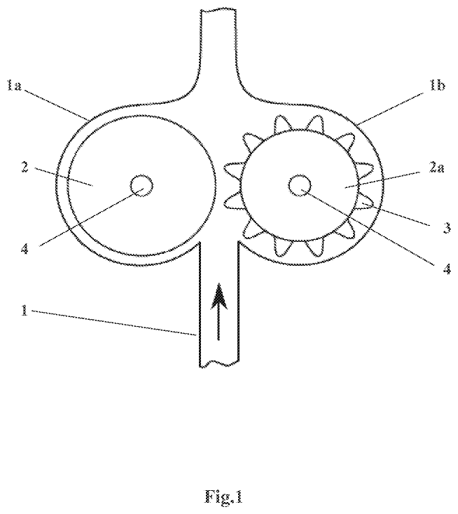

The microporator (FIG. 1) consists of a microchannel 1 with recesses 1a and 1b, equipped with cylindrical (forming surfaces are cylindrical) rollers 2 and 2a. The teeth 3 are made on the forming surface of the roller 2a. The shape of teeth 3 is not very relevant, it is important that their apexes are rounded. The rollers 2, 2a are mounted on axles 4.

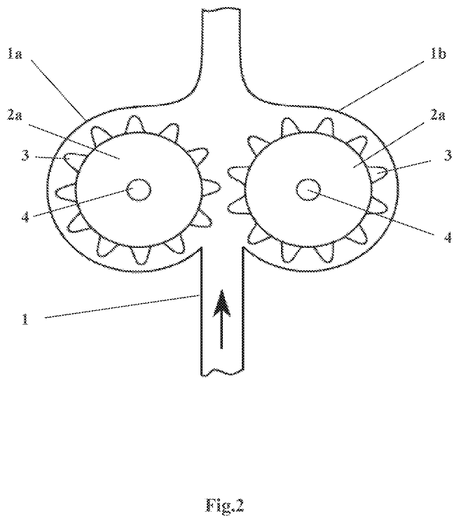

FIG. 2 shows the microporator with both rollers 2a having teeth 3.

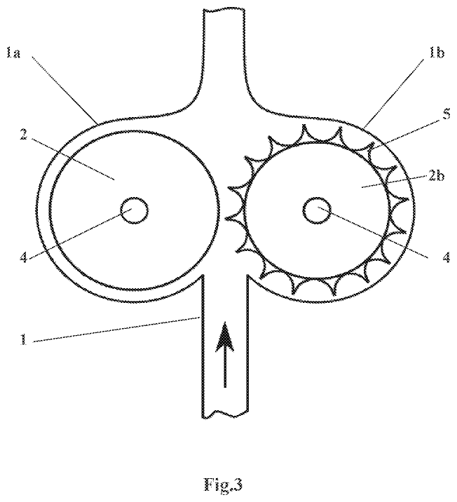

The difference between the microropotor (FIG. 3), similar to that shown in FIG. 1, is that it has a single roller 2b, which has spikes 5 on the composing surface.

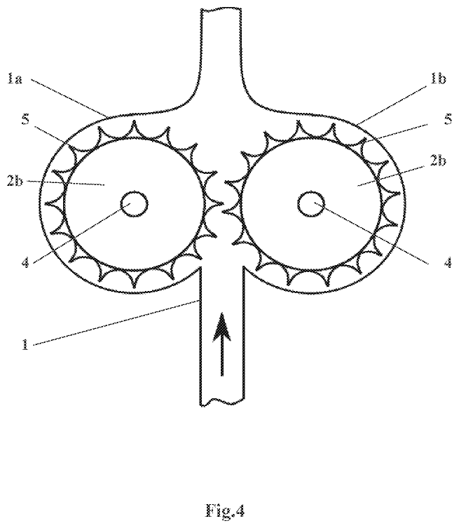

FIG. 4 shows a microporator having two rollers 2a with spikes 5.

The installation of rollers 2, 2a, 2b in microchannel 1 and in other ways is possible. The rollers may have short projections at ends 2, 2a, 2b, while a microchannel may have recessions in walls 1, or conversely, short projections in the walls of the microchannel, while recessions in rollers 2, 2a, 2b, and these are slide bearings.

The following modifications of microporators are possible when the roller pair consists of:
cylindrical and toothed roller with a smooth composing surface,
both toothed rollers,
cylindrical and rollers with spikes with a smooth composing surface,
both rollers with spikes.

Microporators may vary in shapes of roller-composing surfaces. The above-mentioned pairs of rollers had cylindrical composing surfaces, but they may have other ones.

A pair of rollers may have inter-superpose convex-concave surfaces, both concave or inter-superpose cylindrical stepped ones.

In all cases, the gap between the flat composing surface roller 2 and the roller 2a with teeth 3 or the gap of both rollers 2a with teeth 3a, or the gap between the smooth composing surface roller 2 and the roller 2b with teeth 5, or the gap of both rollers 2b with spikes 5 has been selected so that it is smaller than the size of the cells to be processed but not smaller than the dimensions of the non-pressurized small organs (e.g., cell nucleus) or elements present in the cell.

### DETAILED DESCRIPTION OF THE EMBODIMENTS OF THE INVENTION

### Operation of the Microporator

After the microporator has been installed in the transfection device, a liquid medium containing the transport fluid, the cells to be treated and the material to be inserted flow through the microchannel 1 by means of pumps. Passing through the gap between the rollers (FIG. 1, 2), they are deformed, i. e. mechanically compressed, the internal volume of the cells decreases, and after passing through the gap between the rollers 2, 2a, their internal volume recovers, the cells absorb the desired substances through the pores in the membranes.

When microporatators are used in the transfection device (shown in FIG. 3.4), when the cells pass between the pairs of rollers 2, 2b, or 2b, 2b, they are partially deformed and their membrane is punctured by spikes 5. After passing the gap between the roller cells, the inner volume of the cells tries to recover, and they absorb the materials they want to insert through the resulting holes.

Unlike other options of microporators proposed in other known technical solutions, cell slip friction and shear deformation, which is detrimental to cells, are eliminated.

In this case, the cells are only squeezed the gap between the rollers or compressed and the membrane is punctured during the compression.

### Characteristics of Microporators

Indicative dimensions of some elements in microns (μm):
the cross-sectional area of the microchannel—10-100 μm²,
the diameter of the rollers—100-600 μm,
the diameter of axles or axle projections—10-60 μm,
the gap between rollers and microchannel walls—6-20 μm,
the height of the teeth—3 and 20 μm,
the height of the spikes—3-10 μm,
the diameter of the spike tip—0.1-1 μm.

An important parameter of the microporator is the gap, depending on its modification:
the gap between the roller having an even composing surface and the teeth—6-50 μm,
the gap between the roller teeth—6-50 μm,
the gap between the roller with an even composing surface and teeth—6-50 μm,
the gap between the teeth—6-50 μm,
the gap between the rollers and the teeth—6-50 μm.

The cell deformation in the microporator is known to possibly be between 10% and 80% of the average diameter of cells, so a specific microporator must be manufactured for a specific cell type by selecting the above-mentioned modification and gap.

### Manufacture of the Microporator

It can be manufactured in several ways. The first method is when a fluid microchannel, with its recesses for the roller and cavities, can be produced for the bearing elements by mask etching by reactive plasma. The corroding mask may be formed by flat lithography, laser lithography or printing techniques. Another method is laser ablation (which is suitable for transparent, opaque, organic, inorganic materials) or exposure to laser radiation in combination with wet corrosion. For example, wet corrosion can be performed on a transparent tray by exhibiting the tray by means of focused intense short- and ultrashort (femtosecond) pulsed laser radiation, exceeding the material damage threshold and performing a scan, and then treating the tray with a corroding solution. In the case of a glass tray, where the substance is based on silicon dioxide, the corroding agent may be a fluoric acid solution or a hot aqueous solution of potassium hydroxide.

A microchannel can be formed monolithic as a cavity consisting of a tray volume or composed of two layers—the substratum and superstratum. If a combination of two layers is formed, the substratum of the part of the microchannel shall be formed, and the superstratum may be flat and glued or laser welded to the substratum. This action can be called the sealing of the microchannel. Recesses or bearing points may be formed in parallel or consecutively in the same said manner.

Other parts, i.e. roller, gears, spikes, needles, axles, protrusions, bearings, including recesses for bearing points, can be produced in several ways. The most convenient techniques of production are three-dimensional laser lithograph, additive laser lithograph, double photon or multiphoton laser lithograph, laser 3D printing. The essence of the method consists of local exposure of a solution of organic or organic-inorganic monomers or a solid gel, focused by pulsed laser radiation, exceeding the polymerisation threshold dose and inducing a localised polymerisation reaction. Scanning of a focused laser beam can produce derivations of free 3D complex geometry. After production, unexposed material may be removed during the development process with an appropriate solvent. This is a process that does not require assembly and is called non-assembly technology.

The substances which may be used for this purpose are Su-8, Ormocomp, SZ2080, etc.

The most suitable material for this process is SZ2080, which is an organic-inorganic precursor to a polymer designed for laser lithography. Silicon, zirconium, and oxygen-based long inorganic chains with methyl methacrylate groups that interact with each other to form a polymer matrix during nonlinear interactions with laser radiation or in interaction with impurities generated by radicals.

The surfaces of the elements of the microchannel may be further treated to reduce friction and adherence of cells to the walls of the microchannel. The surface of the polymeric SZ2080 3D derivative can be activated with a Piranha solution to form OH-groups on the surface and can be silanized with low surface energy containing fluorosilanes such as (heptadecafluor-1,1,2-2-tetrahydradecyl) dimethylchlorosilane, or add 1h, 2h, 2h-perfluorooctyltriethoxysilane of 5% characterised with superhydrophobic properties to SZ2080, which will conjugate with SZ2080 via —Si—O—Si— bonds during the condensation process. A small amount of this additive does not interfere with photopolymerization and does not wash out after development.

The proposed technical solution for the microporator has the following characteristics:

There are three possible ways for cells to be exposed during transfection: deformation, puncture, and deformation and puncture, the cells are spared during transfection, they are not affected by slip friction and shear deformations, the cells are not affected by side factors during transfection, such as electric, magnetic or acoustic fields, nor they are irradiated.

The process of transfection is very efficient.

1. A cell microporator containing a fluid microchannel (1), containing elements for mechanical action on cells comprising:
mechanically acting elements are made as two rotating rollers, one of which (2) has a smooth composing surface and the other (2a) has teeth (3);
a gap between the constituent surfaces of the two rollers (2.2a) is smaller than the size of the cells to be treated.

2. The microporator according to claim 1, wherein the composing surfaces of the two rollers (2a) have teeth (3).

3. A cell microporator having a fluid microchannel (1), which contains elements for mechanical action on cells, wherein:
the elements for mechanical action are made as two rotating rollers, one of which (2) has a smooth composing surface and the other (2b) has spikes (5) (needles);
the gap between the two rollers (2.2b) is smaller than the size of the cells to be treated.

4. The microporator according to claim 3, wherein the forming surfaces of the two rollers (2b) have spikes (5).

5. The microporator according to claim 1, wherein the forming surfaces of the rollers (2, 2a, 2b) are cylindrical.

6. The microporator according to claim 1, wherein the forming surfaces of the pairs of rollers (2, 2a, 2b) are superposed with each other (convex-concave).

7. The microporator according to claim 1, wherein that the forming surfaces of the roller pairs (2, 2a, 2b) are concave.

8. The microporator according to claim 1, wherein the pairs of rollers (2, 2a, 2b) have stepped, superposed, cylindrical composing surfaces.

9. The microporator according to claim 1, wherein the rollers (2, 2a, 2b) have recesses at the ends and protrusions (short axes) are made in the fluid microchannel (1), which serve as slide bearings.

10. The microporator according to claim 1, wherein the rollers (2, 2a, 2b) have projections at the ends and the fluid microchannel (1) has recesses that serve as slide bearings.

11. The microporator according to claim 1, wherein the rollers are mounted on axles (4) which are mounted on the walls of the fluid microchannel (1).

12. The microporator according to claim 1, wherein the rollers (2, 2a, 2b) are mounted in symmetrical recesses (1a, 1b) of the fluid microchannel (1) and are in one side contact with the cells to be treated.

13. The microporator according to claim 1, wherein the rollers (2, 2a, 2b) in the fluid microchannel (1) are passive and are rotated by a flowing liquid.

14. The microporator according to claim 1, wherein the profile of the teeth (3) is pyramid-shaped with a rounded apex.

15. The microporator according to claim 1, wherein one (2a) of the rollers has teeth (3) and the other (2b) has spikes (5).

16. The microporator according to claim 1, wherein several pairs of rollers can be mounted in the fluid microchannel (1).
